(19) United States
(12) Patent Application Publication
(10) Pub. No.: US 2021/0071127 A1
(43) Pub. Date: Mar. 11, 2021

(54) TRANSPLANTABLE UNIONS OF LIVING TISSUES 3D PRINTED UPON ELECTRONIC CIRCUITS AND OPTICAL SYSTEMS AND THE METHOD OF MAKING THEM -

(71) Applicant: HENRY ANDREW LEVY, BROOKLYN, NY (US)

(72) Inventor: HENRY ANDREW LEVY, BROOKLYN, NY (US)

(21) Appl. No.: 15/931,756

(22) Filed: May 14, 2020

Related U.S. Application Data
(60) Provisional application No. 62/920,750, filed on May 14, 2019.

(51) Int. Cl. C12M 1/42; C12N 5/0775; C12M 3/00

(52) U.S. Cl. CPC C12M 35/02; C12M 21/08; C12N 5/0667

(57) ABSTRACT

Transplantable unions of living cells with electronic circuits and optical systems interfaced with body parts, will be used to test, measure, monitor, control, repair, replace, or assist the functionality of body parts.

Ordinary human cells and stem cells are deposited upon, grown, attached to, united, and coupled with non-rejectable biocompatable electronic circuits, sensors, radio transceivers, optical, electro-optical, and/or computer systems that are designed to be adaptively coupled with the cells to form new transplantable unions. Cells are 3D-printed, deposited, dispensed, painted, sprayed, air-brushed, injected, squirted, spritzed, or delivered by other means upon circuits, or optical systems that are designed and prepared to adaptively couple with the cells.

### PRIOR ART

Deposition, dispensing, painting, spraying, and filling machines are decades old and are ubiquitous worldwide.

Bioprinting human tissue cells today is a well known and widely practiced art. It has progressed very fast and greatly since I wrote, ?Living Tissue Rapid Protyping System? in the Emhart NASA Tech Briefs Create the Future Design Contest in 2006 (Merit Prize Winner), and since I wrote ?Multi Reservoir Airbrush Living Tissue 3D Stem Cell Painter/Injector,? also published in the NASA Create the Future Design Contest in 2014. Today, ordinary human cells and stem cells are cultivated, separated and 3D (three dimensionally) printed by bioprinters on flat or shaped surfaces, structural scaffolds derived from animals, or 3D scaffolds that are 3D printed themselves. Electrodes have long been implanted in the human body and are used to measure or control bodily functions for a hundred years.

Printed circuits and integrated circuits have been manufactured for more than 50 years. These circuits are inserted within or positioned to touch the skin, implanted within the human body, internal body organs, and other body parts.

There are problematic limits to the effective-ness of these procedures. Electronic contact effectiveness for transmitting, receiving, and measuring electrical or electronic signals by surface or embedded contacts are not as good as electrode implantation. Electrodes used in electrencephalographs, polygraphs, and eletrocardiographs are attached to the skin via conductive gels and conductive adhesives. But gel and adhesive conductors do not touch many points per unit surface area. Electrode implantation is labor intensive, destructive, and produces scar tissues which interferes with electrical signal transmitivity. Making scar tissue is part of the body?s methods of self repair. If a body part is cut and then a circuit is introduced to the cut area, scar tissues form at the cut [symbol] area location. Any cut cells, severed organ, or body part will produce scar tissue while repairing itself which greatly interferes with bodily function, electrical conductivity, electrical transmitivity, nerve signal transmission, measurement, and control.

Transplantable unions of selected specialized stem cells and ordinary cells on electronic circuits and optical systems that are designed to adaptively couple with the cells differentiate and grow together with adjacent cells of the body wherever they are transplanted to solve the above described historical problems.

Before this patent, the process of taking human cells, stem cells, and cellular tissues, then depositing them upon, and growing them upon electronic circuits or optical systems, (that were designed to adaptively couple with the cells) and then transplanting the unions along with the attached circuits or optics to humans and animals has not been done in the prior art to overcome the aforesaid problems.

### BRIEF SUMMARY

Transplantable unions of living cells with electronic circuits and optical systems interfaced with body parts, will be used to test, measure, monitor, control, repair, replace, or assist the functionality of body parts.

Ordinary human (or non-human) living cells, stem cells, and other cells are deposited upon, grown, attached to, united, and coupled with non-rejectable biocompatable electronic circuit means, means (145), radio transceiver means, optical, electro-optical, and/or computer system means that are designed to be adaptively coupled with the cells to form new transplantable unions. Cells are 3D-printed, deposited, dispensed, painted, sprayed, air-brushed, injected, squirted, sprinkled, spritzed, or delivered by other means upon electronic sysren means, or optical system means that are prepared to adaptively couple with the cells.

The transplantable union can be part of a cybernetic organism. The circuits can be manufactured by ordinary methods, 3D printing, or by other means. Integrated circuits (ICs), printed circuits, other circuits, and optical system means are designed to adaptively couple with the cells by having surfaces, undercuts, ridges, depressions, spaces, bumps, holes, hooks, and geometric shapes to which living cellular tissues can adhere, or get caught on, within, underneath, and inbetween physically and frictionally. These tissues, as well as, wire attached circuits and/or optical systems upon which the tissues are grown, attached, combined with, and, coupled with, form a union which is then transplanted into the human body or into an organ that is temporarily outside of the body.

Using this method, the optic nerve, the retina, the spinal cord, the brain, the heart, lungs, liver, kidneys, cochlea, pancreas, thyroid, blood vessels, lymph system, muscles, skin, and other human body organs or parts can be connected to, interfaced with , and combined with electronic system means and optical systems means. These unions, or coupled interfaces with human body parts, are used to measure, monitor, control, repair, replace, or assist the functionality of body parts, and for many other purposes.

### DESCRIPTIONS OF PATENT FIGURES AND LIST OF FIGURE PART NUMBERS AND THEIR DESCRIPTIONS

Patent Figure Descriptions:

FIG. 1

3D Cell printer/dispenser deposits ordinary cells and stem cells on a micro-grid array of an electronic circuit, sensors, and a microprocessor integrated circuit

FIG. 2

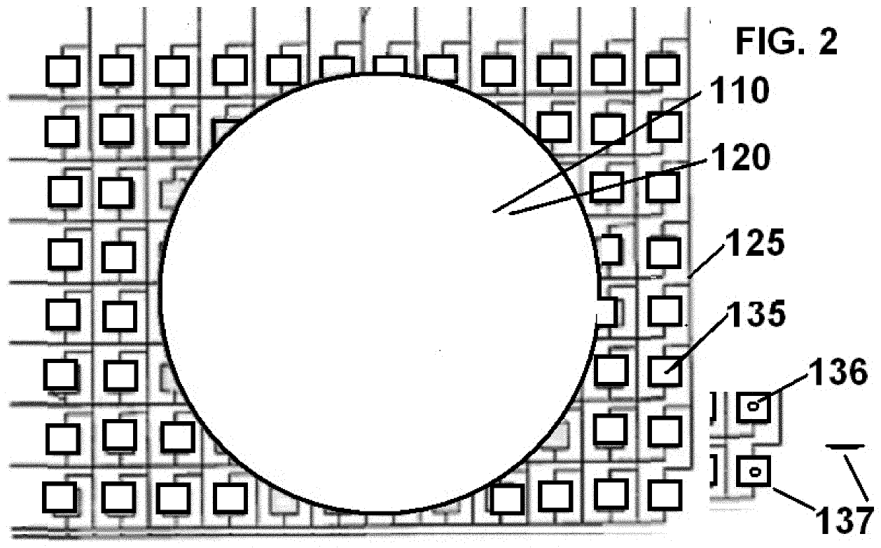

Stem cells and ordinary cells printed upon a micro-grid array of electronic circuit electrical contacts before trimming and implantation

FIG. 3

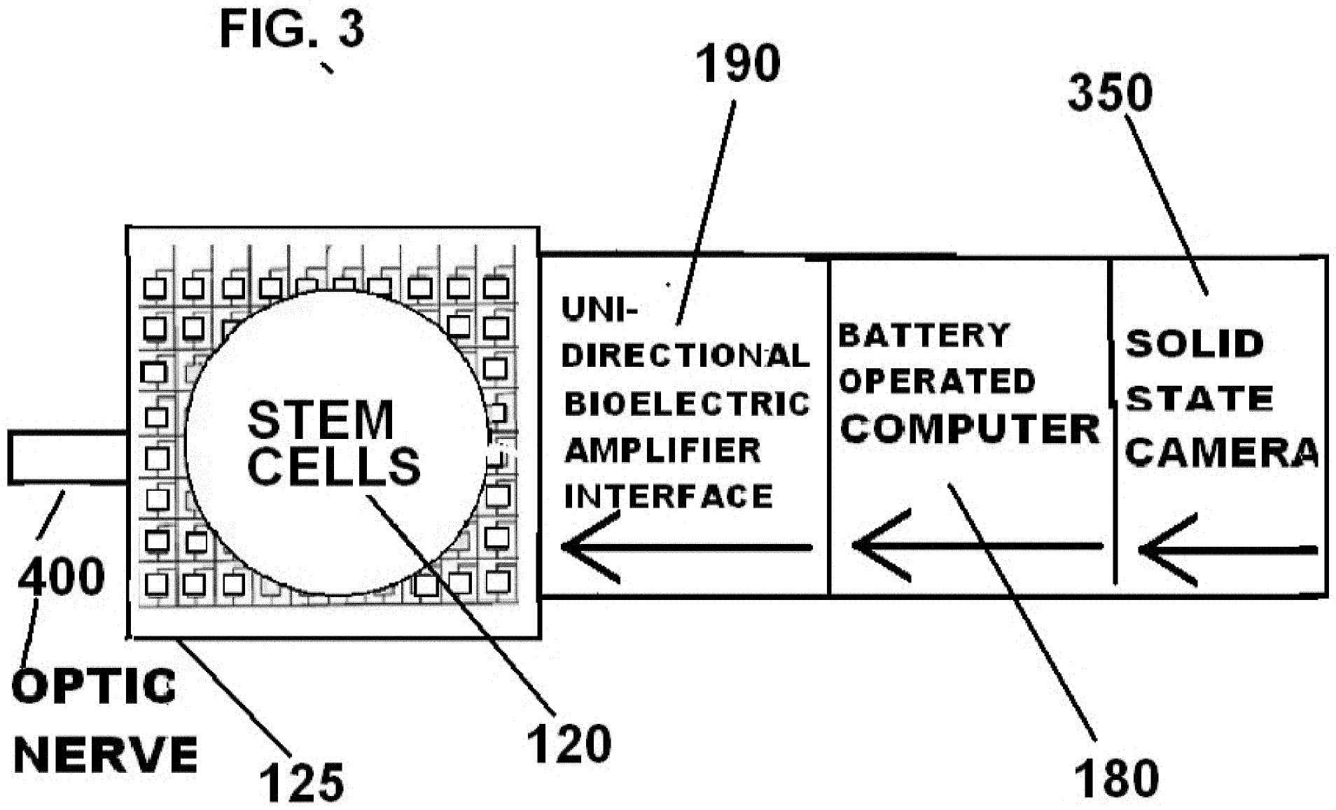

Eye Replacement:

Functional block diagram of:

Electronic signals representing camera images are transmitted to a computer which transmits electrical signals to a union of stem cells printed upon a micro-grid-array of electrical contacts of an electronic circuit implanted within a severed optic nerve to restore vision to a blind person

FIG. 4

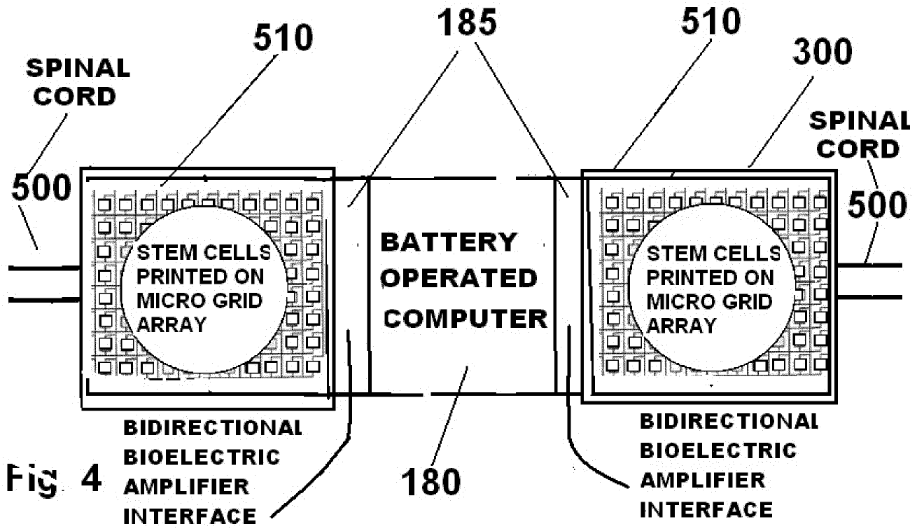

Spinal Cord Repair:

Functional block diagram of:

Electrical signals from severed spinal cord nerves are amplified and transmitted bidirectionally through unions of stem cells and micro-grid arrays of electronic systems through a computer to enable severe spinal cord injury repair.

FIG. 5

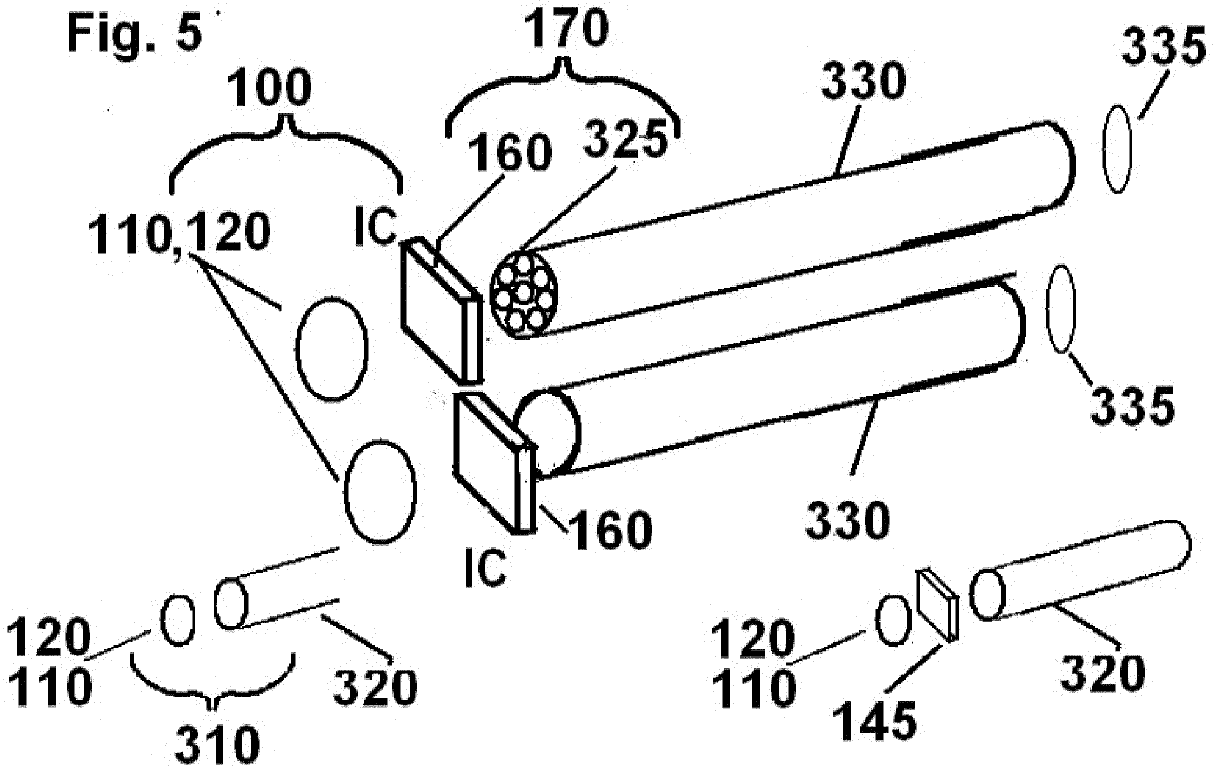

Stem cells on an IC contacting a coherent fiber optic bundle, and separately contacting one fiber optic fiber, and stem cells directly contacting an ordinary fiber optic fiber

### LIST OF ELEMENT PART NUMBERS OF TRANSPLANTABLE UNIONS BIOPRINTER PATENT DRAWINGS May 10, 2020

1OO Transplantable unions of living cells with electronic system means (100)

310 Transplantable unions of living cells with optical system means

115 electronic system means

315 Optical system means

110 Ordinary living human tissue cells

120 stem cells

125 ordinary cells and stem cells 3D printed upon a micro-grid array

135 electrical contact of grid of contacts

136 electrical contact with through hole

137 electrical contact with undercut

140 non-rejectable electronic substrates

145 sensors

150 non-rejectable 3D printed circuit boards

160 non-rejectable 3D integrated circuits

[symbol] and microprocessors

170 electro-optical system

180 computer

185 bidirectional bioelectric amplifier

190 unidirectional bioelectric amplifier

[symbol] interface

200 reservoir of cells

210 dispensor nozzle

215 cell injector needle

220 deposition machine that 3D prints cells with replaceable printing head, sprayer nozzle, and needle injector head and liquid cell solution dispensing means

225 sprayer nozzle

320 optical fibers

325 interstitial spaces between optical fibers

330 coherent fiber optic bundles

400 optic nerve cells

500 spinal cord

510 stem cells on spinal cord

### DETAILED SUMMARY

Transplantable unions (100) of living cells with electronic system means (115) and optical system means (300) interfaced with body parts, will be used to test, measure, monitor, control, repair, replace, or assist the functionality of body parts. Ordinary living human tissue cells (110) (or other tissue cells) and stem cells (120) are deposited upon, grown upon, attached to, united with, and coupled with non-rejectable biocompatable electronic system means (115), and electro-optical system means (170) that are designed to be adaptively coupled with the cells to form new transplantable unions. Cells are 3D-printed, deposited, dispensed, painted, sprayed, air-brushed, injected, squirted, spritzed, or delivered by other means upon circuits, or optical systems that adaptively couple with the cells. Electronic system means (115) are biocompatable non-rejectable electronic substrates (140), non-rejectable 3D printed circuit boards (150), non-rejectable 3D integrated circuits (160). non-rejectable 3D electronic semiconductor circuit scaffolds, discrete electronic [symbol] components,] microprocessors (160), controllers, transceivers, computers (180) other electronic circuit means, optical fibers (320), electro-optical system means (170), lasers, mirrors, LED's coherent fiber optic bundles (330).

Today typically, ordinary living human cells (120) and stem cells (110) are collected from a human. Similarly, cells can be taken from animals for use with animals. These cells are cultivated, nurtured, incubated, multiplied. and grown. These cells are then nurtured in an incubator or other conducive, protective environment. They are then separated and put into liquid solutions by commonly known existing methods. The liquid solutions of cells are put in reservoirs (200) connected to liquid cell solution dispensing means (220) also by commonly known existing methods. In this invention, the cells are then printed, 3D printed, deposited, dispensed, painted, sprayed, spritzed, air-brushed, injected, atomized, thrown, sprinkled, dropped, dripped, flung, squirted, or delivered by other means upon target electronic circuit means (100), electro-optical system means (170), or optical systems means. (315) Once delivered, [symbol] These cells become flat and/or 3D shaped deposited cellular tissues (220) attached to, coupled with, combined with, and united with the target electronic circuit means (115), electro-optical system means (170), and/or optical system means. Stem cells (120) have biochemical interactions with adjacent cells, differentiate and become the same as adjacent ordinary cells and body cells. These cellular tissue cells (110) (120) grow together to become body tissues. The tissues grow upon, grow into, and grow within the surfaces, undercuts, through holes and the shapes of the electronic system means (115) and/or electro-optic system means, thereby attaching themselves to the electronic or optical systems to become coupled with and united with them. Layers of deposited scaffolds and.or adhesives can also assist adhesion or sticking to the above said system means. The tissues that have coupled with the electronic system means (115), optical system means (315), and/or electro-optic system means (170), are then transplanted to the human body (or organs not yet in a body) (or into other life forms) where they continue to grow and attach themselves to similar cellular tissues in the body. The transplanted cells (310) including stem cells (120) attach themselves and grow together with similar cells at the transplant site. The stem cells (120) bio-chemically interact with adjacent ordinary cells and differentiate to become like their adjacent ordinary cells. Electronic circuits (115) are connected by non-rejectable biocompatable wires or fiber optics that are threaded through and exit the body. Alternatively, the circuits are wire connected to implanted radio means, radio transceiver means, radio transmitter means, or just radio receiver means. Radio means can be WIFI, bluetooth, zigbe, or other radio means with replaceable or inductively charged rechargeable batteries.

Adhesion

The components composing the circuits and the circuit layers of the electronic circuit means, and (330), and electro-optical circuit means can be built to have undercuts, depressions, bumps, ridges, holes, hooks, edges, and geometric shapes to which living cellular tissues can adhere and get caught on, within, and inbetween physically and frictionally thereby multiplying cellular adhesion to the circuits. Interstitial spaces (350) between optical fibers will provide cellular adhesion to cells bioprinted and grown upon them. The structures of the electronic circuit means, the optical system means (300), and the electro-optical system means (170) can be further adapted to be coupled and configured to adhere to the living cells that are deposited upon and into them. Scaffold layers can be printed upon the electronic circuit means, the optical system means (315), and the electro-optical system means (170) to increase adhesion of living cells to them. Non-rejectable adhesives can also be used to make cells stick or adhere to circuits means and/or optical system means (315). Such adhesives and/or scaffolds can be electrically conductive or optically transparent. Thousands of electrical contact points to the tissue cells are possible. Contact points can also have aforementioned undercuts to assist adhesion, connectivity, conductivity, and electrical signal transmitivity.

The human cells (110) and human stem cells (120) are harvested. collected, and separated by known processes and then put into solutions in reservoir containers (200).

These reservoir containers or modules are refillable, permanent, or replaceeable. Commonly, the reservoirs (200) are refillable with ordinary specific human cells (110) or mixtures with stem cells (120) and other materials somewhat like ink is put into inkjet refillable or replaceable inkjet cartridge modules of an inkjet color printer. Other kinds of human or animal cells, as well as other materials are also placed in the same or other reservoir cartridge modules. Growth factors, collagen, antibiotics, fluids, and additional kinds of materials (as later described), are placed in their own separate reservoir container modules that are replaceable or refillable. Typically, reservoirs in bioprinting machines (220) are connected to a variety of different kinds of individual printing, deposition, and dispensing means [symbol] (210). Individual deposition means that are used are either dispensing nozzles, inkjet printer modules, sprayers, spritzers, sprinklers, ultrasonic atomizers, or other known means of delivering cells to a specified locations such as other cells, body organs, or scaffolds. Cells are usually deposited upon a flat surface or plattens, but in this invention, cells and other materials are dispensed upon and into printed circuit boards (150), semiconductor discrete electronic components, semiconductor scaffolds, integrated circuits (160), other electronic circuit means, optical system means (315), and electro-optical system means (170) for the purpose of permanent attachment. Unique in this patent, is that computer controlled cell dispensing (deposition) is upon electrical, electronic, and/or flat surfaces, shaped 3 dimensional surfaces, on a semiconductor scaffold, discrete electronic components, electro-optical components (170), surface mount components, printed circuit boards (150), on integrated circuits (IC's) (160) and aforementioned electronic system means. Deposition can also be on individual fiber optic fibers (320) fiber optic bundles (330), lenses (335), lasers, mirrors, or other optical system means (315). Reservoir modules in some bioprinters have individual tubes leading to dispensing means or the modules themselves may have built in dispensing means. These are similar to very common fluidic deposition systems in use for decades depositing glue, soldier paste, ink, paint, food, cake frosting, ice cream, candy, and a myriad of other materials.

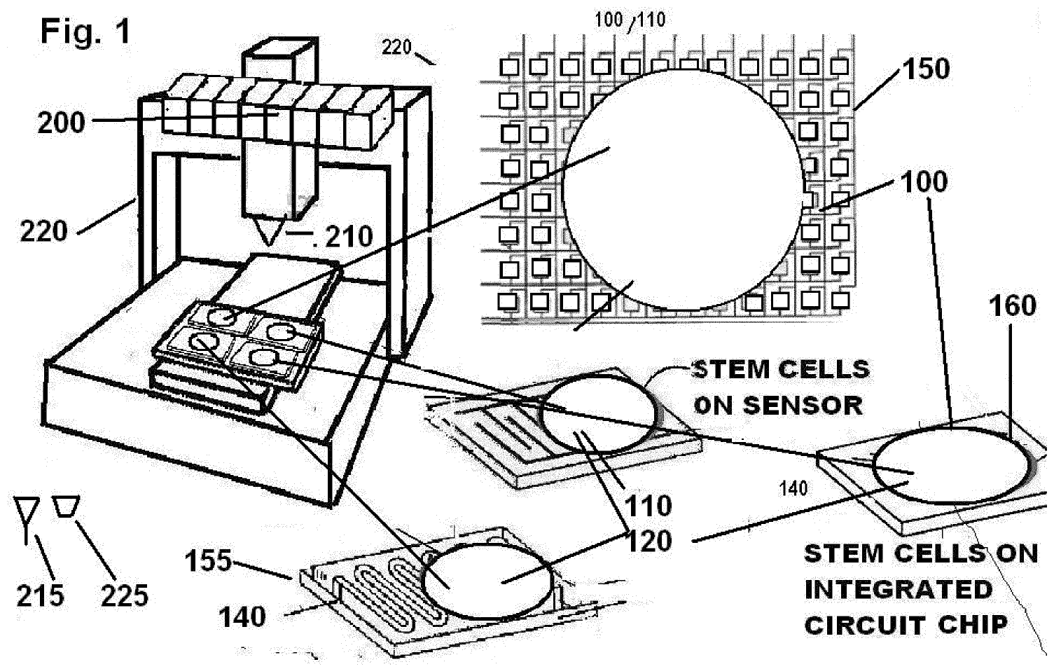

Commonly, either the dispensing means move over the dispensing target, or the dispensing target is moved under the dispenser or both move. Dispensing means (210) are moved in XYZ preprogrammed patterns moving left and right, or up and down, over a target surface. In the figure of the dispensing machine (FIG. 1), the dispensing means dispensing living tissue cells and other said materials move over and deposit upon the target surfaces which are electronic system means (115), electro-optical system means (170), and/or optical system means (315). They are moved by stepper motors, servo motors, fractional horsepower Ac, DC, or 3-phase motors driving linear translation stages, rotary stages, linear motors, gantry XYZ positioning systems, robotic systems, or other very well known existent factory automation deposition systems.

The deposition or dispensing head (210), needle (215), or sprayer head (225) is moved in pre-programmed graphic patterns dispensing and depositing living cells and other materials on optical, electrical, or electronic surfaces to create living tissues on them. The cells are deposited on the surfaces in preplanned programmed graphic patterns, in one to thousands of cycles creating layers. All electrical, electronic, electro-optic, or optical components to be combined with living cells can be selected from those materials that are biocompatable and not rejectable by the body or are encased in material that is not rejectable, and are those materials that do not produce an antigenic response by the body or are otherwise toleratable.

Cells can be delivered upon electronics by preprogrammed bioprinters, preprogrammed automatic sprayers, preprogrammed robotic dispensers, preprogrammed sprayer/injectors, or other aforementioned means.

Sprayer Injector

A handheld or robot held airbrush stem cell sprayer pen (head) is used to selectively airbrush spray OR INJECT cells, ordinary cells (110), stem cells (120), other cells, and other ingredients onto (or into) electronic structures, 3D printed circuits, 3D integrated circuits, optical systems, and semiconductor scaffolds in thin film layers. Cells can be put onto or into electronic substrates (140), non-rejectable 3D printed circuit boards (0150), non-rejectable 3D integrated circuits (160), non-rejectable 3D electronic semiconductor circuits scaffolds, optical fibers (320), coherent fiber optic bundles (330), electro-optical systems (170), lasers, lenses (335), or other electronic component means encased in non-rejectable enclosures with non-rejectable contact means, other circuit means and systems.

The sprayer/injector can also be used to selectively airbrush spray or inject stem cells (120), other cells, and other ingredients onto (or into) injured human tissues, incubating cultured living tissues, and dissolvable 3D preprinted scaffolds for growing living tissues in thin film layers. This handheld airbrush stem cell sprayer pen (or injector) can be guided by a human hand or preprogrammed computer vision guided robotic end effector. Essentially, whether it is in pen shape or any other shape, it is a fluidic dispenser and/or injector head. Several cooled reservoirs feeding the sprayer/injector pen are filled with autologous cells, mesenchymal stem cells (120), allogenic cells, dermal fibroblasts, xeogenic cells, syngeneic or isogenic cells, primary cells, secondary cells, adult and embryonic stem cells (120), tissue-matrix materials, biochemical growth factors, collagen, blood, multipotent, pluripotent, and totipotent stem cells (120). Induced pluripotent stem cells (120) (iPSCs) are ordinary adult cells that have been genetically reprogrammed to be in an embryonic-stem-cell-like state. Such genetic reprogramming is achieved by adding genes or changing genes of the chromosomes of the cell to become like embryonic stem cells' chromosomes. Some of the same materials are also selectively put in previously described reservoirs (200) of liquid solutions [symbol] (210) of all bioprinting and cell deposition systems. Materials to print scaffolds can also be put in the reservoirs (200).

A controlling computer can select location, the [symbol] sequence, and quantity of cells to be deposited.

The multi-fluid reservoirs (200) selectively deliver to a printer head, painter head, sprayer head, nozzle, injector pen, airbrush sprayer head (or injector (215) head) the chosen cell type (or other material) to be sprayed or dispensed upon a target objective. In this invention, the target objectives to deposit cells upon are selected from any one of the following targets: integrated circuits (160), printed circuit boards (150), optical systems, semiconductor scaffolds, other aforementioned electronic circuit means (130), [symbol] 5] (300), dissolvable scaffolds for vascular grafts, skin grafts, kidney replacement grafts, pancreas islet cell replacement grafts, liver cell replacement heart muscle replacement grafts, and bone or cartilage replacement grafts. All grafts, [symbol] deposition systems can also be used to deposit sequential layers of cells on the following targets: fiber optic fibers 320), lasers, lenses (335), or coherent bundles of fibers (320), other electro-optical systems (170), as well as, onto or into a whole organ on a dissolvable scaffold.

Deposition/Sprayer/Injector/Heads (250):

Some of the many kinds of interchangeable replaceable printer heads, deposition heads, and painter pen airbrush sprayer/injector heads used are described as follows:

Cells are dispensed through nozzles, sprayers, spritzers, air-brush heads, squirters, and, atomizers controlled by preprogrammed automatic motorized motion control systems or manually. Replaceable reservoirs (210) of ordinary tissue cells (110), stem cells (120) or (other above described materials) are used [symbol] in the most bioprinters and the handheld painter pen airbrush sprayer head. Reservoirs (200) are like the replaceable cartridges in Hewlett Packard or Cannon color inkjet printers or a hypodermic syringe without a needle . A configuration of the hand held pen, has direct or programmable selector buttons similar to a selectable soda dispenser in a bar or soda fountain shop. The buttons select the materials or cells o be sprayed or deposited. Selector buttons can be in the hand held sprayer or remote to it.

Sprayer:

Compressed air from a computer controlled compressor forces cells and other fluidic materials from the selected reservoir through tubes to the sprayer pen head. The compressed air is selectively activated or switched on computer controlled electropneumatic valves or electrohydrolic valves which drive the fluids containing cells through the nozzle sprayer. Water cleanser is thrust through the tubes and pen while pointed towards waste disposal between selections to clean it.

Another configuration is the same as either above system except that an injector needle (215) replaces the sprayer head. The injector needle (215) can inject ordinary cells (110) and/or stem cells (120) into areas, spaces, undercuts, and cavities in electronic structures, ICs, and semiconductor scaffolds. It can also deliver stem cells (120) and other cells to tissues killed in cancer treatments or in other areas to stimulate growth.

Another configuration is a robotic arm's end effector dispensor whose preprogrammed controller directs the dispensing and deposition of cells onto a target.

In this way, the multi-reservoir airbrush living tissue 3d stem cell painter/injector sprayer pen can be used to paint with living tissues onto electronic structures, onto 3D printed scaffolds, onto ICs, onto PC boards, and onto semiconductor scaffolds, and other aforementioned electronic circuit means. Many other kinds of deposition dispenser nozzles can be used in bioprinters or pens. Ordinary bioprinters or bioprinter pens can also be used to replace burned or otherwise damaged tissues, deposit tissues, build tissue grafts, build organs, and ultimately to save lives.

All of the above described heads can be used in a standard bioprinters, biodispensers, xyz positioning systems, gantry xyz positioning systems, cartesian and polar coordinate robotic systems.

One or thousands of thin film cellular layers can be put on or into electronic systems by repetitive cycles of printing\spraying\deposition\injecting.

Advantages

There are many uses of electronics and optics with cells deposited, [symbol] [grown, and attached to them which are then transplanted to a human or animal body. For example, if optic nerve cells (400) were harvested, grown, cultivated, separated, and then 3D printed along with optic nerve stem cells (410) upon an integrated circuit (160), printed circuit (150), or semi-conductive scaffold, electrical signals from the electronic circuits could transfer signals to the optic nerves. This would be more effective than electrode implanting the optic nerve with fine wires with resulting destructive scar tissues, and more effective than severing the optic nerve (400) bundle and placing circuits upon the severed section with its similar scar tissue formation. Stem cells (120) deposited on scaffolds help repair an optic nerve (400) or spinal cord, One could replace the eye or bypass the eye with an electronic camera (350) and a 3D printed interface (composed of 3D printed cells and 3D printed battery-powered integrated circuits (160)) between the camera electrical signal output and interface to the optic nerve. This would be done rather than trying to accomplish the same result by electrode implantation or electrical multicontact implantation, thereby, getting greater resolution from the camera communicated to the brain through the optic nerves than previously achieved. Stem cells (100) mixed with the layers of optic nerve cells (400) deposited on an integrated circuit (160) would all together be transplanted onto the body's optic nerve. The airbrush sprayed or injected stem cells, other cells, and other ingredients deposited onto (or into) electronic circuit means (115), along with the circuits upon which they were placed, cultivated and grown are transplantable. Those transplantable cells and circuits (100) when transplanted will then grow together with and attach themselves to he body's optic nerve. The stem cells (120) will differentiate, and “know” by chemical interactions with adjacent optic nerve cells (400), that the stem cells (120) should grow and multiply to produce optic nerve cells (400). And, when these are 3D printed on dense integrated circuit contacts 160) (150), it is possible to electrically stimulate optic nerve cell fibers with electrical signals derived from transplanted electronic circuits (100) upon which cells had been deposited, grown, and transplanted. Such circuits would be connected to an implanted electronic camera (350) (with a rechargeable battery) which replaces the eye or a camera that is external to a non-working eye. Batteries can be charged inductively periodically.

Although some spinal cord injuries might be repaired by printing stem cells (120) upon silicone or other scaffolds and transplanting them to the spinal injury site, some very destructive spinal injuries leaving large gaps and missing areas can in the future be connected by electrical connection by the the following method: Spinal cells (510) and stem cells (120) are deposited or printed on two separate integrated circuits (160). See FIG. [symbol] . The deposited cells and the integrated circuits (160) are transplanted to the two severed ends of the spinal cord (500). The two integrated circuits (with spinal cord cells (510) and stem cells (120) at each end of the severed spinal cord (500) would be connected to each other by wires or optical fibers (320) bridging the gap that resulted from the spinal injury. Integrated circuits (160) would communicate by wires, optical fibers (320), or coherent fiber optic bundles (330) to each end's electronic circuits interface coupling to the ends of the spinal cord. The circuits will be connected to and powered by an inductively charged rechargeable battery that is implanted within the body, printed on the circuits, is part of the circuits, or is external to the body.

Electrical signal communication to muscle fibers cells can also be improved via this invention. Electrical signals to muscle fibers produce electro myofibrylic responses (muscle contractions) or contraction of muscle myofibrils. Muscle fiber cells are grown along with stem cells (120) upon densely printed non-rejectable printed circuits (150) or integrated circuits (160). These are transplanted to selected muscles with attached wires, threaded internally through the body to the spinal cord (500), nerves, or external electronic controllers which can control the muscles.

And, in another example, organs of the body such as the heart or pancreas can be attached directly to electrical contacts which have high contact density to make measurements or to stimulate the organ. Measurement of glucose in the blood, hydration, or other blood characteristic might be improved. Heart behavior measurements , or heart electrical stimulation could also be improved.

Auditory nerves can be printed on electronic circuits that have electronic amplifiers that then communicate directly to auditory nerves.

Electrical contacts to brain parts in rats, monkeys, or humans can be improved over traditional electrode implantation by 3D printing brain cells and brain stem cells directly on densely-packed integrated circuits (160) or non-rejectable biocompatable printed circuit boards (150) or integrated circuits (160) to better communicate electrical signals to the brain cells or to tap brain signal electrical signals. Electrical signals from the human brain can be detected for the purpose of measurements or thought recognition. Brain waves can be monitored more precisely and accurately. Electrical stimulation of the brain can also be done more effectively than previously. This can be done via printed brain cells with stem cells (120) attached to non-rejectable integrated circuits (160) or non-rejectable printed circuits (150). Non rejectable 3D printed circuits cell contacts could be printed with titanium via ion sputtering, vacuum deposition, or laser etching. The human tissues would be grown, separated, and 3D printed upon the electronic interface circuits which could also be shaped and be 3D printed. First the electronic circuits would be made. Then, [symbol] [the human tissues would be printed upon them or injected into them. Then the electronic circuits (with attached wires) with the cells upon them would be transplanted to the human.

These tissues grown upon electronic circuits and optical system means will interface with human body organs and parts more effectively than by electrode implantation or by placing electronic circuits upon, next to, or within human body tissues alone.

The density per unit surface area of electrical contact points on human cells (110) (120) are greatly increased by 3D printing the cells upon integrated circuits (160) or finely 3D printed circuit boards' micro-grid arrays (125). Growing human cells (110) and stem cells (120) after 3D printing them on flat, shaped, or 3D printed electronic circuits contact points with undercuts or through holes (125), semiconductor scaffords, silicone scaffolds, or upon integrated circuits 160) (possibly in an incubator environment) have several other advantages after they are transplanted to a human or animal. Some other advantages are that living cells taken from the body, fabricated organs, and removed organs can be printed on [symbol] electronic chemical testing circuits (145) which monitor, test, measure, and control functional characteristics of the organ after transplantation back into the body. Skin cells can also be printed on electronic or electrical circuits and transplanted to the body. Human cells (110) (120) that are are printed on chemical sensors (145) that measure glucose levels, sodium levels, and [symbol] hydration in organs and blood vessels can be transplanted. WiFi, Bluetooth, other transmitters, or transceivers powered by inductively charged rechargeable batteries can be implanted which when connected electrically to transplanted electronic circuits with cells bioprinted upon them will communicate collected data to external displays, monitoring devices, or phones. Muscles can be controlled by preprogramed controllers via implanted contacts and circuits. Transcutaneous nerve stimulation can be improved with transplanted electronic contacts in limbs to control the limb's muscles enabling people to walk with computer controlled muscle stimulation. Pain control management can be improved with similar implants. Signal transmission from the heart and brain can be improved for many purposes. Human cells (110) (120) can be bioprinted upon pressure sensors, temperature sensors, image sensors, chemical sensors (145), and other sensors.

Vital signs such as pulse rate, blood pressure, oxygen levels, and body temperature can be monitored via sensors (145) (comprised of cells bioprinted [symbol] upon sensors (145) which are implanted within a human or animal. Implanted chemical sensors (145) can monitor carbon dioxide levels, hormone production, ph, acidity, and other body function characteristics.

Other advantages are avoidance of scar tissue growth of both cut cellular bodily tissues and in tissues with implanted electrodes. More useful effective electrical contacts (125) with undercuts and through holes can be made to healthy cells than can be made to scarred cells. This improves electrical signal transmission within and through the tissues. Another advantage is better adhesion of bodily cellular tissues to the electronic or electrical surfaces due to undercutting and geometric holding structures produced by 3D printing.

The structures of the electronic circuit means, the optical system means (315), and electro-optical system means (170) can be further adapted to be coupled to living cells that are deposited upon and into them. Brain neural implants, memory implantation, and means of uploading and downloading memories can be facilitated and improved. Artificial intelligence circuit chips can be implanted into the brain to enhance thinking or repair motor functions. Thus there are very many new advantages of transplantable unions (100) of 3D printed ordinary cells (110) and stem cells (120) onto electronic circuit means (130) and optical system means (315).

1. A transplantable union of living tissue cells with optical system means (selected from at least one of the following group of optical system means: (optical fibers, coherent fiber optic bundles, fiber optic interstitial spaces. lasers, LEDs, lights, lenses, mirrors, sensors, cameras, and other optical systems) united by cell deposition means, wherein, deposited living cells are attached to, connected to, combined with, grown upon, and coupled with the structure of optical system means.

2. A transplantable union of claim 1 wherein the cells deposited are both ordinary cells and stem cells.

3. A transplantable union of claim 1 wherein the cells deposited are only ordinary cells.

4. A transplantable union of claim 1 wherein the cells deposited are only stem cells.

5. A transplantable union of claim 1, wherein, living cells injected into parts of the optical system means (rather than by only having been deposited upon them) attach the living cells to the optical system means.

6. A transplantable union of claim 1 wherein the deposited cells are attached to, connected to, combined with, and coupled with the structure of an optical system means that is designed to be adapted to be coupled with the cells that are deposited upon them.

7. A transplantable union of living tissue cells and electronic system means selected from at least one of the following group of electronic system means: (electronic contact means, substrates 3D printed circuit boards, integrated circuits, 3D electronic semiconductor circuit scaffolds, discrete electronic components, surface mounted components, electro-optical components, controllers, sensors, computers, transceivers, receivers, transmitters microprocessors, and other electronic systems means) by cell deposition upon the electronic system means wherein at least one region of the electronic system means is prepared to couple with the deposited cells.

8. A transplantable union of claim 7, wherein the design of the electronic system means assists in the adherence of the deposited cells to the electronic system means.

9. A transplantable union of claim 7, wherein the electrical contacts of the electronic circuit means are shaped to have undercuts built into them that hold the cells deposited onto them improving the electrical conductivity and signal transmitivity of the electrical contacts to the cells.

10. A transplantable union of claim 7, wherein the electrical contacts of the electronic circuit means have electrically conductive adhesive on them that hold the cells deposited onto them, to their contacts, and attach, combine, couple, and electrically connect the contacts with the cells.

11. A transplantable union of claim 7, wherein,
living cells injected into parts of the electronic system means (rather than by only being deposited upon them) attach the living cells to the electronic system means.

12. A transplantable union of claim 7 wherein the cells deposited are both ordinary cells and stem cells

13. A transplantable union of claim 7 wherein the cells deposited are only ordinary cells

14. A transplantable union of claim 7 wherein the cells deposited are only stem cells.

15. A transplantable union of claim 7 wherein no region of the electronic system means is prepared to couple with the deposited cells to increase adherence of the cells to the electronic system means, rather instead, the cells are deposited upon the unprepared electronic system means.

16. A transplantable union of claim 7 wherein at least one kind of living cells selected from the following group of living cells: (ordinary cells, ordinary stem cells, spinal cord cells, optic nerve cells, brain cells, auditory nerve cells, and retinal cells) are deposited upon and united with at least one system from the following group of systems: (electronic system means, optical system means, and electro-optical system means).

17. A method of making a transplantable union of living cells and electronic system means wherein living cells are deposited, attached to, connected to, combined with, grown upon, united with, and coupled with the structure of an electronic system means, wherein at least one region of the electronic system means is prepared to couple with the deposited cells increasing the adherence of the cells to the electronic system means.

18. A method according to claim 17 wherein electrical contacts of the electronic system means are shaped to have undercuts built into them that hold the cells deposited onto them improving the electrical conductivity and signal transmitivity of the electrical contacts to the cells.

19. A method according to claim 17 wherein, conductive adhesive is applied between the the electronic system means and the living cells deposited on electronic system means to increase adherence of the cells to the electronic system means and their contacts

20. A method according to claim 17 wherein living cells are injected into the electronic system means, rather than only being deposited onto the electronic system means.
